(19) United States
(12) Patent Application Publication
(10) Pub. No.: US 2024/0192188 A1
(43) Pub. Date: Jun. 13, 2024

(54) METHOD OF AND AN APPARATUS FOR DETERMINING DIETARY FIBER AND A SAMPLE CONTAINER SYSTEM FOR USE THEREIN

(71) Applicant: Foss Analytical A/S, Hilleroed (DK)

(72) Inventor: Thomas PALM, Hilleroed (DK)

(73) Assignee: Foss Analytical A/S, Hilleroed (DK)

(21) Appl. No.: 18/554,881

(22) PCT Filed: Apr. 5, 2022
(86) PCT No.: PCT/IB2022/053135

§ 371 Date: Oct. 11, 2023

(30) Foreign Application Priority Data
May 19, 2021 (DK) PA202100522

(51) Int. Cl. G01N 33/02; G01N 1/28; G01N 1/44

(52) U.S. Cl. CPC G01N 33/02; G01N 1/286; G01N 1/44; G01N 2001/2866; G01N 2333/926; G01N 2333/95

(57) ABSTRACT

A sample container system for use in a method for determining dietary fiber comprises a sample container and a magnetic mixer. The sample container has a floor and a rigid side-wall upstanding therefrom to together delimit a sample containing space, wherein the floor includes a porous filter portion. The magnetic mixer has at least one concentrically arranged rotor-stator, each of the at least one concentrically arranged rotor-stator including a rotor and a stator, the rotor and the stator defining therebetween an annular shear gap, the rotor configured to rotate relative to the stator, the stator including a stator body having a first plurality of openings therethrough, the rotor including a magnetic coupling and a rotor body having a second plurality of openings therethrough, the magnetic coupling configured to couple with and follow an externally generated rotating magnetic field to cause the rotor to rotate in relation to the stator.

### CROSS-REFERENCE TO RELATED APPLICATIONS

This application is a National Stage of PCT/IB2022/053135, filed on Apr. 5, 2022, which claims priority to Danish Patent Application PA202100522, filed on May 19, 2021 in the Danish Patent and Trademark Office, the entire contents of each of which are incorporated herein in their entirety.

### BACKGROUND

The present invention relates to a method of and an apparatus for determining dietary fiber and to a sample container system for use in the same. In particular the invention relates to the determination of total dietary fiber.

The determination of the dietary fiber content of food for animal consumption (commonly referred to as ‘crude fiber’ content) is a valuable aid in the prediction of its nutritional value to the animals. The level of dietary fiber provided by food for human consumption is also important to determine as dietary fiber is known to benefit human health by aiding digestion and helping to prevent heart disease. For these reasons at least, food manufacturers invest research and resources into means of optimising the dietary fiber content of their products and it is therefore important for the industry to have reliable and accurate means of measuring this dietary fiber.

Many methods exist for the determination of dietary fiber in both human and animal food such as, for example methods approved by the Association of Official Analytical Chemists (AOAC) like, “Total, Soluble, and Insoluble Dietary Fiber in Foods” (AOAC Method 991.43) and “Analysis of Crude Fiber in Feed” (AOAC 978.10). Generally such methods are enzymatic/gravitation methods and involve the steps of dissolving non-fiber components in a neutral or acid detergent solution or an acid solution followed by alkaline solution, typically whilst undergoing heating and stirring; recovering the residual insoluble dietary fiber fraction by filtration and washing; and determining the weight of the recovered dietary fiber after drying. The determination of total dietary fiber involves an additional step of precipitating the soluble fiber fraction using alcohol and recovering this precipitate by filtration and weighing. Additionally, according to the known methods for dietary fiber determination, the so determined weight(s) needs to be corrected for protein and ash content of the sample which is also present in the solids that are isolated from the solution(s) after the dissolving and optional precipitation steps.

Such methods typically require weights to be determined with an accuracy of ±0.005 g. It is therefore important that essentially all of the dietary fiber obtained from the dissolving and the optional precipitation steps described above is made available for weighing.

To this end, it is known from, for example U.S. Pat. No. 9,182,382, to provide a sample container for use in the determination of dietary fiber consisting of a flexible reaction/filter chamber made of a combination of porous and non-porous materials and divisible into separate compartments by means of a releasable seal. The chamber is configured with an open first end; an opposing second end which is formed of a porous material; and a flexible side-wall of non-porous material connecting the two ends. By this container the problematic transfer of mixtures between beakers and filters is eliminated and thus the problem of loss of recovered fiber between such transfers is mitigated. However, transfer of sample mixture from one compartment to another is still required which may lead to some material remaining in the first compartment and thus lost to any further processing in the second and subsequent compartments.

A method of analysing a sample to determine a dietary fiber content is also disclosed in U.S. Pat. No. 9,182,382 and utilizes the sample container disclosed in that document. The method comprises placing the sample into a chamber of the disclosed container; weighing the combined sample and container to obtain a first weight; reacting the sample in the chamber with one or more enzymes in solution whilst heating and stirring to obtain an insoluble dietary fiber fraction; allowing the solution to pass through the filter and into a second container similar to the first and precipitating out a soluble dietary fiber fraction by adding an alcohol solution to the solution in the second container; filtering the precipitation solution through the filter of the second container; and drying and weighing both the insoluble and the soluble dietary fiber fractions in the two containers.

### SUMMARY

It is an aim of the present invention to alleviate at least one of the problems associated with the know systems and methods for determining dietary fiber.

According to a first aspect of the present invention there is provided a sample container system comprising a sample container having a floor and a rigid side-wall upstanding therefrom to together delimit a sample containing space; wherein the floor includes a porous filter portion and wherein the sample container system further comprises a magnetic mixer having at least one concentrically arranged rotor-stator, with the rotor and stator of each concentrically arranged rotor-stator forming therebetween an annular shear gap and having the rotor mounted for rotation relative, the stator including a stator body provided with a plurality of openings, for example slots, such as notches, therethrough and the rotor including a magnetic coupling, such as a bar magnet or a magnetic material, and a rotor body provided with a plurality of openings, for example slots, such as notches, therethrough. The magnetic mixer is only associated with an own sample container to form an inseperate unit, thereby eliminating or minimizing the possibility of changes to the volume or composition (through contamination) of a sample within the sample containing space that can result when introducing atmospheric external mixing elements into the sample. Moreover, the rotor-stator arrangement, when operated, can provide a more homogenous distribution of particulate sample in liquid within the sample container which may speed up or increase repeatability of any chemical reaction between the particulate sample and the liquid.

In some embodiments the rotor is provided with a plurality of blades extending beyond the stator body in a direction along the rigid side-wall away from the floor and rotatable with the rotor body. These help move material contained in the containing space towards the rotor body to thereby improve mixing.

In some embodiments one or both the rotor and the stator are provided as inserts for the sample container. In some of these embodiments the stator may be an insert formed with a portion of an outer surface of its stator body adapted to engage with an inner surface of the rigid side-wall.

In some embodiments the stator may have a portion of an outer surface of its stator body permanently attached to an inner surface of the rigid side-wall. This permits a simplified manufacture of the sample container and the stator as a single entity, such as by using plastics moulding technology.

According to a second aspect of the present invention there is provided a method for determining a dietary fiber content of a sample, such as a food sample, the method comprising: providing a sample container; placing a sample into the sample container and obtaining a first weight; performing an enzymatic digestion of the sample in the sample container, preferably under mixing; separating a solid residue from liquid in the sample container by filtration through the porous filter section of the sample container; drying the sample container and solid residue; obtaining a second weight; calculating a difference between the first and the second weights; and determining the dietary fiber content of the food sample dependent on the calculated difference; wherein the sample container is a sample container according to the first aspect of the present invention and wherein the first and the second weights consist of the combined weights of the sample container, including the magnetic mixer, and its contents. Thus, a single container is employed so that problems associated with loss of material during transfer between containers is removed. Moreover, the use of an integrated magnetic mixer further mitigates loss of any recovered fiber since the mixer remains within the sample container and any fiber and other solid matter deposited on to it remains to be weighed.

In some embodiments there is provided a step of adding an alcohol solution to the sample following the step of performing enzymatic digestion and before the step of separating a solid residue, preferably while stirring, to precipitate out a soluble dietary fiber fraction from the liquid in the sample container.

In some embodiments the step of separating the solid residue from liquid in the sample container comprises a step of generating a first pressure gradient across the porous filter portion in a direction to create a lower pressure below the filter portion outside the sample container than inside the sample container. This helps speed up filtration.

Operating the mixing device during the step of separating the solid residue from liquid in the sample container to ensure movement of solid residue and liquid thereby achieving no or minimal risk of clogging of porous filter section and subsequent more reliable and fast filtration.

In some embodiments the step of reacting the sample in the sample container with at least an enzymatic solution comprises a step of generating a second pressure gradient across the porous filter portion in a direction to create a higher pressure below the filter portion outside the sample container than inside the sample container during the step of reacting the sample in the sample container. This helps prevent egress of liquid through the filter.

In some embodiments the mixing device may be used to speed up drying step, after filtration step, by ensuring movement of solid residue to continuously expose moist parts of the solid residue.

Most usefully, the method also comprises a step of determining a weight of protein and a weight of ash in the solid residue in the sample container and the step of determining a dietary fiber content comprises correcting the second weight for the weights of protein and of ash.

To determine ash and protein the dried residue of the sample container is emptied. Operating the mixing device prior to empty of sample container may be done to easy removal of residue, as the mixing device mechanically release residue from inner geometry of sample container.

According to a third aspect of the present invention there is provided an apparatus for determining a dietary fiber content of a food sample, the apparatus comprises a number of sample container systems; a plurality of liquid reservoirs selectively fluidly connectable with the sample containers of the number of sample container systems to deliver liquid thereto; and a controller operably connected to at least the plurality of liquid reservoirs and adapted to control the operation of the apparatus to perform the method according to the second aspect of the present invention wherein each of the number of sample container systems consists of a sample container system according the first aspect of the present invention and wherein the apparatus further comprises a magnetic drive means magnetically couplable to the magnetic coupling of each of the number of sample container systems and operable to generate a rotating magnetic field to rotate the rotor.

In some embodiments the magnetic drive means comprises a mechanically rotatable bar magnet located beneath the floor of each sample container of the number of sample container systems.

In some embodiments the magnetic drive means comprises a stator located about an external periphery of the rigid side-wall of each sample container of the number of sample container systems and having electrical windings energisable to create a rotating magnetic field.

### BRIEF DESCRIPTION OF THE DRAWINGS

These and further advantages, modifications and embodiments of the present invention will now be further described with reference to the accompanying figures, of which:

FIG. 1 Shows a schematic representation of a first embodiment of a sample container system according to the first aspect of the present invention;

FIG. 2 Shows a schematic representation of a second example of a magnetic mixer of a sample container system according to the first aspect of the present invention;

FIG. 3 Shows a schematic representation of a third example of a magnetic mixer of a sample container system according to the first aspect of the present invention;

FIG. 4 Shows a flow-chart representation of an embodiment of the method according to the second aspect of the present invention;

FIG. 5 Shows a schematic representation of an embodiment of an apparatus according to the present invention; and

FIG. 6 Shows a second example of a magnetic drive means of an apparatus according to the present invention.

### DETAILED DESCRIPTION

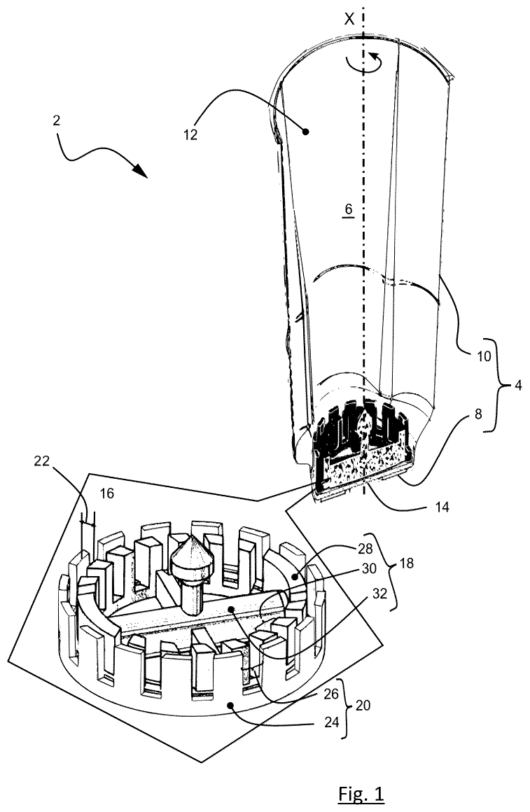

A first embodiment of a sample container system 2 according to the present invention is illustrated in FIG. 1. The sample container system 2 comprises a sample container 4 which has an elongate sample containing space 6 delimited by a floor 8 and a long side-wall 10 which upstands from the floor 8 and which terminates in an open end 12. The long side-wall 10 is formed of a rigid, non-porous material, such as polypropylene or polyester. The floor 8 is provided with a porous filter portion 14 which, in some embodiments, may form the entire floor 8. The porous filter portion 14 is, in the present embodiment, also hydrophobic in nature. Being hydrophobic in nature helps control the retention of liquid within the sample containing space 6 for a time necessary to allow digestion and precipitation to occur, as described below. The sample container system 2 further comprises a magnetic mixer 16 which, in use is located within the sample containing space 6 and which forms an inseparate unit with the sample container 4. Thus a magnetic mixer 16 of the present invention is associated with only one sample container 4.

The magnetic mixer 16 is of the known rotor-stator type and is illustrated in more detail in the exploded view of FIG. 1. Generally, rotor-stator mixers comprise an impeller or rotor rotating in close proximity to a stationary housing or stator. As it rotates, the rotor draws in sample from above and/or below, expels it through openings in the rotor and mechanically imparts high-shear forces to it. With rotor-stator mixers, clumps of particles are also broken apart by the hydraulic shearing forces generated as they are ejected through openings in the stator into the bulk of the sample. Thus, the stator directs the flow and confines the particles while the rotor imparts shear.

The magnetic mixer 16 comprises a concentrically arranged rotor 18 and stator 20 with the rotor 18 and the stator 20 being separated along the diagonal by an annular shear gap 22. The stator 20 has a stator body 24 in which are formed a plurality of openings, here notches 26 through which sample in the sample containing space 6 will be forced by the rotation of the rotor 18. The rotor 18 comprises a rotor body 28 in which are formed a plurality of openings, here notches 30, typically corresponding with the notches 26 of the stator body 24, through which sample in the sample containing space 6 can pass into the annular shear gap 22. In the present embodiment the stator 20 is fabricated as a part of the sample container 4, for example the sample container 4 and the stator 20 may be fabricated from a suitable plastic material by a moulding process, and the rotor 18 is fabricated, for example from a suitable plastic material, as an insert to this sample container 4 to be located concentrically with the stator 20. In other embodiments both the rotor and stator are fabricated separate from the sample container 4 and both provided as inserts to the sample containing space 6 of the sample container 4. However formed, each rotor-stator 18-20 arrangement is associated with only one sample container 4. The magnetic mixer 16 and the sample container 4 are therefore considered to be an inseparate unit.

A magnetic coupling is also provided as a part of the rotor 18 and here comprises at least one (here one) bar magnet 32 or other magnetic material which, in some embodiments, may be detachably mounted to the rotor body 28 so that it may be removed, cleaned and re-used. The magnetic coupling 32 is configured to couple with and follow an externally applied rotating magnetic field which rotates the rotor 18 about an axis X through the common centre of the concentrically arranged rotor 18 and stator 20 in a plane that is generally parallel to the to the floor 8.

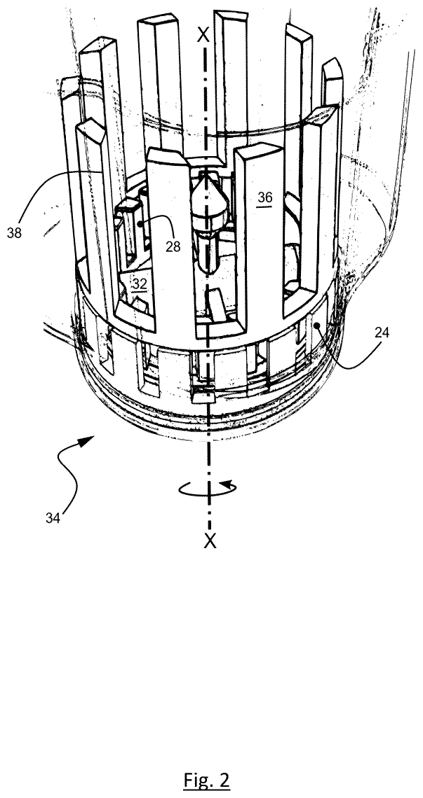

A second example of a magnetic mixer 34 of a sample container system according to the present invention is illustrated in FIG. 2, in which the elements in common with the magnetic mixer 16 of FIG. 1 are given the same reference numerals. As with the magnetic mixer 16, the magnetic mixer 34 comprises a concentrically arranged rotor 18 and stator 20 with the rotor 18 and the stator 20 being separated along the diagonal by an annular shear gap 22. The rotor body 28 is, in this second example, additionally provided with a plurality of blades 36 that extend beyond the stator body 24 in a direction along the rigid side-wall 10 of the sample container 4. These blades 36 help mix the bulk of sample in the sample containing space 6 as the rotor 18 rotates. In some embodiments the plurality of blades 36 each have at least leading edges 38 which are located sufficiently close to, and in some embodiments in contact with, the rigid side-wall 10 and which, as the rotor 18 rotates, further act to scrape away sample that adheres to the side-wall 10.

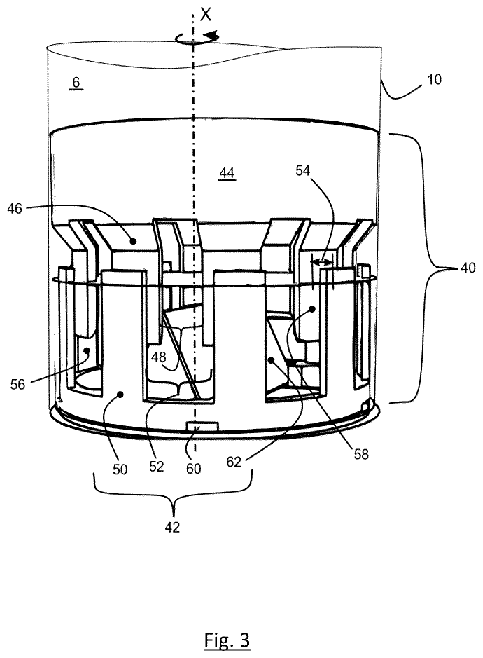

A third example of a magnetic mixer 40 of a sample container system according to the present invention is illustrated in FIG. 3. The magnetic mixer 40 comprises a concentrically arranged rotor 42 and stator 44. The stator 44 has a stator body 46 in which are formed a plurality of openings, here notches 48, and the rotor 42 comprises a rotor body 50 in which are formed a plurality of openings, here notches 52. In this example, the stator body 46 is located closer to the common center of the concentrically arranged rotor 42 and stator 44 than the rotor body 50 so that an annular shear gap 54 is formed between an internal surface 56 of the rotor body 50 and an external surface 58 of the stator body 46. In the present example, lugs 60 on the rotor body 50 provide a reduced contact area between the side-wall 10 and the rotor body 50 and allows the rotor 42 to more easily rotate within the sample containing space 6.

A magnetic coupling is also provided as a part of the rotor 42 and here comprises at least one (here one) bar magnet 62 or other magnetic material. The magnetic coupling 62 is, in common with the other examples of the magnetic mixers described above, configured to couple with and follow an externally applied rotating magnetic field to rotate the rotor 42 about an axis X which passes through the common centre of the concentrically arranged rotor 42 and stator 44.

For purposes of illustration only, an exemplary embodiment of the method according to the present invention will now be described in relation to the method for determining the total, soluble and insoluble dietary fiber in foods according to the AOAC 991.43 method (the contents of which are contained herein by reference). This requires that duplicate samples of dried foods, fat-extracted if containing >10% fat, undergo sequential enzymatic digestion by heat stable α-amylase, protease, and amyl-glycosidase to remove starch and protein. For total dietary fiber (TDF), enzyme digestate is treated with alcohol to precipitate soluble dietary fiber (SDF) before filtering and TDF residue is washed with alcohol and acetone, dried, and weighed. For insoluble and soluble dietary fiber (IDF and SDF), enzyme digestate is filtered, and residue (IDF) is washed with warm water, dried and weighed. For SDF, combined filtrate and washes are precipitated with alcohol, filtered, dried, and weighed. TDF, IDF, and SDF residue values are corrected for protein and ash.

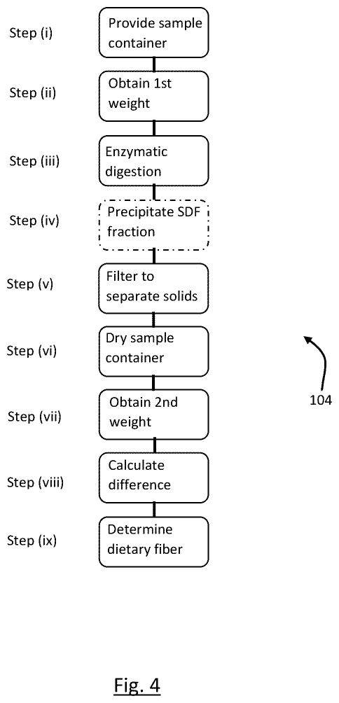

With reference to FIG. 4, a method 104 for determining dietary fiber content of a food sample comprises: providing a sample container system 2 according to the first aspect of the present invention, Step (i); placing the food sample into the sample containing space 6 of the sample container 4 and obtaining a first combined weight of food sample and sample container 4, Step (ii); performing enzyme digestion for a time sufficient to establish a liquid digestate and a solid residue by adding enzymes in solution to the sample containing space 6 containing the food sample and stirring using the integral magnetic mixer 16; 34; 40 (typically heating also), Step (iii); optionally precipitating an SDF fraction from the enzyme digestate in the sample containing space 6 of the sample container 4, Step (iv); separating the solid residue and the SDF fraction in the sample containing space 6 from liquids in the sample containing space 6 by filtration through the porous filter portion 14 of the floor 8 of the sample container 4, Step (v); drying the contents of the sample containing space 6, Step (vi); obtaining a second combined weight of solids and sample container 4 (including the integral magnetic mixer 16; 34; 40), Step (vii); calculating a difference between the first combined weight and the second combined weight, Step (viii); and determining the dietary fiber content in dependence on the calculated difference, Step (ix).

In some embodiments filtration at Step (v) may be enhanced by establishing a pressure gradient across the porous filter portion 14 in a direction to enhance transportation of liquid from inside the sample container 4 to outside the sample container 4.

In some embodiments the Step (viii) of calculating the difference between the first combined weight and the second combined weight comprises determining protein and ash content of the sample and correcting the second combined weight for the determined protein and ash content.

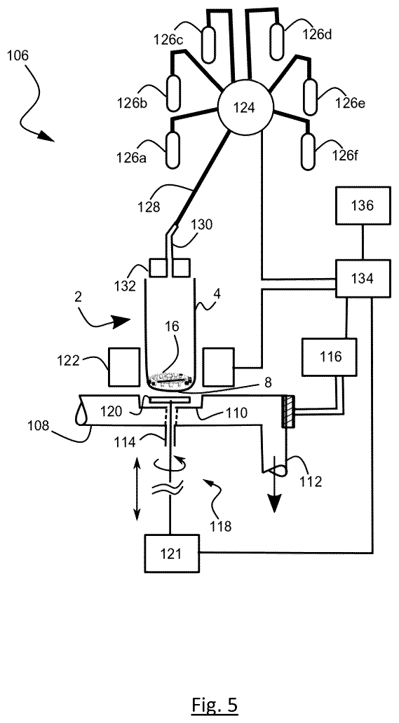

An embodiment of an apparatus 106 according to the third aspect of the present invention is illustrated in FIG. 5 for the automated application of the method 104 according to the second aspect of the present invention which was described above. The apparatus 106 comprises a number (here one) of sample container systems 2 according to the present invention. For ease of description reference will be made in this embodiment, by way of example only, to the sample container system 2 which is illustrated in FIG. 1 and described above. A fluid manifold 108 of the apparatus 106 is configured with a number (here one) of lower interfaces 110 which receives and releasably retains a corresponding sample container 4 of the sample container system 2 and establishes a fluid tight connection between the floor 8 of the sample container 4 and internal of the fluid manifold 108. The fluid manifold 108 has an outlet 112 to waste and a passageway 114 connected with each interface 110. In some embodiments each interface 110 may include a valving system which is operable to control the flow of liquids from the sample containing space 6, through the porous filter portion 14 of the floor 8 of the interfaced sample container 4 and into the fluid manifold 108. The apparatus 106 of the present embodiment additionally comprises an optional pump 116 connected to the fluid manifold 108 and which is operable to at least generate less than ambient pressure (under-pressure) in the fluid manifold 108 to aid in filtration as described below. In some embodiments the pump 116 is operable to also generate above ambient pressure (over-pressure) in the fluid manifold 108 to help retain material in the sample container 4 for a sufficient time to permit digestion and/or precipitation.

A magnetic drive means 118 is provided for each of the number of sample container systems 2 which is operable to generate a rotating magnetic field that couples to the magnetic coupling (bar magnet 32) of the rotor 18 to rotate the rotor body 28. In the present embodiment the magnetic drive means 118 comprises a bar magnet 120 which is mechanically coupled to a rotational drive motor 121 and which is located beneath the floor 8, outside of the sample container 4.

A temperature regulator 122 of known construction, which in some embodiments may comprise a heater unit and in other embodiments may comprise a heater/cooler unit, is operable to regulate the temperature, for example to heat, the number of sample containers 4. A multi-way valve 124 is configured to selectively couple of a one of a plurality of liquid reservoirs (here six illustrated) 126a-126f with a conduit 128 that connects with an inlet 130 of an upper interface 132 of a number of upper interfaces (here one illustrated). For example, water 126a, ethanol 126b, acetone 126c, sodium hydroxide 126d, hydrochloric acid 126e and enzyme solution(s) 126f may be contained in an own one of the plurality of reservoirs 126a-126f. Each of the number of upper interfaces 132 is configured to direct transfer of liquid through the open end 12 of a corresponding sample container 4 into the sample containing space 6 and in some embodiments forms a fluid tight connection therewith. A controller 134 is provided to control the operation of the apparatus 106 to automatically perform a determination of dietary fiber content of a food sample, for example according to the AOAC 991.43 method.

In use, an operator inserts a pre-weighed sample container system 2 containing a food sample, typically 1 g±0.005 g, into the associated lower interface 110 and manually connects the associated upper interface 132 to the open end 12 of the sample container 4 of the sample container system 2. In some embodiments connection with one or both of the lower interface 110 and the upper interface 132 may be done automatically using mechanical elements known in the art. In some embodiments the operator may be prompted to enter the weight of the pre-weighed sample container system 2 and food sample into the apparatus 106 via a user interface (not shown) of an associated data processor 136 as a first combined weight. In other embodiments this first combined weight may be transferred automatically into the data processor 136, for example the sample container 4 may include a machine readable label on which this weight is previously stored. The label is of a known type and may be a bar code or radio frequency identity (RFID) chip which will be automatically read by the apparatus 106 using a corresponding reader (not shown) of known type and the previously stored weight provided to the data processor 136 as the first combined weight.

Once the desired number (here one illustrated) of sample containers 4 of the sample container systems 2 are inserted into the apparatus 106 the user may initiate the automatic fiber content determination for example by pressing “Start” on the user interface. On initiation the controller 134 of the present embodiment operates to control the multi-way valve 124 to transfer liquid from selected reservoirs 126a-126f as appropriate during the determination; the temperature regulator in order to establish and maintain a predetermined temperature in the sample containing space 6 of the sample container 4; the magnetic drive means 118 to rotate the rotor 18 of the associated one of the number of sample container systems 2 and stir the contents of the associated sample container 4; the pump 116 to optionally create an over-pressure in the fluid manifold 108 which, via the lower interface 110, generates a pressure gradient across the porous filter portion 14 of the floor 8 of the sample container 4 in a direction to inhibit the flow of liquids from the sample container 4; the pump 116 to optionally generate an under-pressure in the fluid manifold to enhance the flow of liquids from the sample container 4, through the porous filter portion 14. In some embodiments the controller 134 is configured to also control the components of the apparatus 106 to effect flushing of the sample container 4 after precipitation or enzymatic digestion, depending on whether TDF or insoluble dietary fiber (IDF) fractions are to be determined for the food sample.

In some embodiments, the controller 134 is further configured to control the components of the apparatus 106 to rinse the contents of the sample container(s) 4 with acetone from a one of the reservoirs 126c and to activate the temperature regulator 122 to dry the residue within the sample container 4. The sample container system(s) 2 may then be removed from the apparatus 106 and weighed on a weighing device to obtain a second combined weight which is the weight of solid residue in the sample container 4, including the magnetic mixer 16, and which is provided to the data processor 136, for example as user input via a user interface or transmitted to the data processor 136 as digital information generated by the weighing device. In other embodiments a weighing device may be included as a component of the apparatus 106 and the second combined weight may then be obtained within the apparatus 106. Duplicate samples are used to determine in a known manner the protein and ash content of the food sample and provided to the data processor 136. The second combined weight is corrected for ash and protein content in the data processor 136. The weight of dietary fiber in the food sample is then calculated in the data processor 136 dependent on a difference between the first combined weight and the so corrected second combined weight.

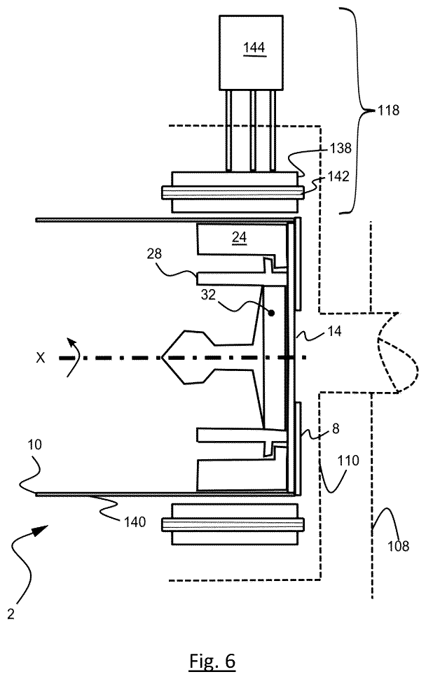

A second example of a magnetic drive means 118 of the apparatus 106 according to the present invention is illustrated in FIG. 6 and comprises an annular stator 138 of generally known construction which is located around an external surface 140 of the rigid side-wall 10 of each sample container 4 of the number of sample container systems 2 in a plane containing the magnetic mixer, say the magnetic mixer 16 illustrated in FIG. 1. The stator 138 has electrical windings 142, for example 3-phase windings, that are energisable to create a rotating magnetic field.

It will be understood by those skilled in the art that the foregoing contains description of exemplary embodiments only of the present invention and that various changes in detail may be made, elements omitted and combinations of various elements of these embodiments may be done without departing from the invention as defined by the appended claims.

1. A sample container system, comprising:
a sample container having a floor and a rigid side-wall upstanding therefrom to together delimit a sample containing space, wherein the floor includes a porous filter portion; and
a magnetic mixer having at least one concentrically arranged rotor-stator, each of the at least one concentrically arranged rotor-stator including a rotor and a stator, the rotor and the stator defining therebetween an annular shear gap, the rotor configured to rotate relative to the stator, the stator including a stator body having a first plurality of openings therethrough, the rotor including a magnetic coupling and a rotor body having a second plurality of openings therethrough, the magnet coupling configured to couple with and follow an externally generated rotating magnetic field to cause the rotor to rotate in relation to the stator.

2. The sample container system of claim 1, wherein at least one of the first plurality of openings or the second plurality of openings are slots.

3. The sample container system of claim 2, wherein the slots are notches.

4. The sample container system of claim 1, wherein the rotor includes a plurality of blades extending beyond the stator body in a direction along the rigid side-wall.

5. The sample container system of claim 1, wherein the magnetic coupling includes a plurality of bar magnets.

6. The sample container system of claim 1, wherein at least one of the rotor or the stator is separate from the sample container and is configured to be an insert for the sample container.

7. The sample container of claim 1, wherein the stator is an integral part of the sample container such that the sample container and the stator are separate portions of a same piece of material.

8. A method for determining a dietary fiber content of a food sample, the method comprising:
a) providing the sample container system of claim 1;
b) placing the food sample into the sample container of the sample container system and obtaining a first weight that is a combined weight of the sample container of the sample container system, the magnetic mixer of the sample container system, and the food sample;
c) performing an enzymatic digestion of the food sample in the sample container and causing the rotor of the magnetic mixer to rotate in relation to the stator of the magnetic mixer to establish a liquid digestate and a solid residue within the sample container subsequently to determining the first weight;
d) separating the solid residue from liquid in the sample container based on filtration through the porous filter portion of the sample containers such that the solid residue remains within the sample container;
e) drying the sample container and the solid residue while the solid residue remains within the sample container;
f) obtaining a second weight that is a combined weight of the sample container of the sample container system, the magnetic mixer of the sample container system, and the solid residue while the solid residue remains within the sample container;
g) calculating a difference between the first weight and the second weight; and
h) determining the dietary fiber content of the food sample dependent on the calculated difference.

9. The method of claim 8, further comprising:
precipitating out a soluble dietary fiber fraction from the liquid in the sample container based on adding an alcohol solution to the liquid in the sample container subsequently to performing the enzymatic digestion and prior to performing the separating of the solid residue from the liquid in the sample container.

10. The method of claim 8, wherein the calculating the difference includes
determining a weight of protein and a weight of ash in the solid residue in the sample container,
correcting the second weight for the weight of protein and weight of ash to determine a corrected second weight for the weight of protein and the weight of ash to determine a corrected second weight, and
determining a difference between the first weight and the corrected second weight.

11. An apparatus for determining a dietary fiber content of a food sample, the apparatus comprising:
a sample container system, including
a sample container having a floor and a rigid side-well upstanding therefrom to together delimit a sample containing space, wherein the floor includes a porous filter portion, and
a magnetic mixer having at least one concentrically arranged rotor-stator, each of the at least one concentrically arranged rotor-stator including a rotor and a stator, the rotor and the stator defining therebetween an annular shear gap, the rotor configured to rotate relative to the stator, the stator including a stator body having a first plurality of openings therethrough, the rotor including a magnetic coupling, and a rotor body having a second plurality of openings therethrough, the magnetic coupling configured to couple with and follow an externally generated rotating magnetic field to cause the rotor to rotate in relation to the stator;
a plurality of liquid reservoirs selectively fluidly connectable with the sample container of the sample container system to deliver liquid thereto; and
a controller operably connected to at least the plurality of liquid reservoirs, the controller configured to control the apparatus to perform the method of claim 8,
wherein the apparatus further includes a magnetic drive means located external of the sample container system and configured to be magnetically couplable to the magnetic coupling of the magnetic mixer of the sample container system.

12. The apparatus of claim 11, wherein the magnetic drive means comprises a rotatable bar magnet configured to be located beneath the floor of the sample container of the sample container system.

13. The apparatus of claim 11, wherein the magnetic drive means comprises a separate stator having electrical windings energisable to create a rotating magnetic field couplable to the magnetic coupling of the sample container system.
